(19) United States
(12) Patent Application Publication
(10) Pub. No.: US 2025/0073061 A1
(43) Pub. Date: Mar. 6, 2025

(54) THERMAL SYSTEM WITH SIPHON DRAINING

(71) Applicant: Stryker Corporation, Kalamazoo, MI (US)

(72) Inventors: Gary L. Bartley, Kalamazoo, MI (US); Christopher John Hopper, Kalamazoo, MI (US); Venkata Ravi Varma Mudunuri, Kalamazoo, MI (US); Megan G. Mackellar, Vicksburg, MI (US)

(21) Appl. No.: 18/724,961

(22) PCT Filed: May 10, 2023
(86) PCT No.: PCT/US2023/021662

§ 371 Date: Jun. 27, 2024

Related U.S. Application Data
(60) Provisional application No. 63/341,634, filed on May 13, 2022.

(51) Int. Cl. A61F 7/00

(52) U.S. Cl. CPC A61F 7/0085; A61F 2007/0018; A61F 2007/0039; A61F 2007/0054; A61F 2007/0093; A61F 2007/0095

(57) ABSTRACT

A thermal control unit includes a supply circuit, a return circuit, a fluid channel, a drain coupled to the fluid channel, and at least one hose circuit connecting the supply circuit and the return circuit. A supply valve may be coupled to the supply circuit and a return valve may be coupled to the return circuit. When the drain is opened, the return valve can open to allow ambient air to enter the return column and to create a siphon to drain the fluid in the thermal control unit. The supply valve can open after the return valve and allow ambient air to enter the supply circuit. The supply valve may allow less air to enter the thermal control unit than the return valve to preserve the siphon through the draining process.

### BACKGROUND

The present disclosure relates to a thermal control system for controlling the temperature of circulating fluid that is delivered to one or more thermal pads positioned in contact with a patient.

Known thermal control units may include a fluid reservoir, a supply circuit, a return circuit, and at least one hose circuit connecting the supply circuit and the return circuit. The thermal control unit adjust the temperature of the fluid within the thermal control unit and pumps that fluid to one or more thermal pads wrapped around the patient. The pads either remove heat from the patient or deliver heat to the patient. After passing through the thermal pads, the fluid returns to the thermal control unit where its temperature is again adjusted in order to heat or cool the patient.

After the thermal control unit is done being used, it typically includes a drain valve positioned at a relatively low position on the unit that, when opened, allows the fluid to drain out of the thermal control unit. The present disclosure relates more particularly to the draining of the thermal control unit.

### SUMMARY

The present disclosure is directed to a thermal control unit that automatically creates a siphon for draining the fluid from the thermal control unit. The automatic siphon creation helps better remove fluid from inside of the hoses used in the thermal control system. This better removal of fluid reduces the labor required by the technician to drain the hoses, and helps reduce the likelihood of bacteria growth and/or the growth of other microbes.

According to a first aspect of the present disclosure, a thermal control unit is provided for controlling a patient's temperature during a thermal therapy session. The thermal control unit includes a first fluid port adapted to fluidly couple to a first hose and a first fluid column having a top end fluidly coupled to the first fluid port. The first fluid column having a first internal volume. The thermal control unit includes a second fluid port adapted to fluidly couple to a second hose and a second fluid column having a top end fluidly coupled to the second fluid port. The second fluid column having a second internal volume greater than the first internal volume. The thermal control unit includes a fluid channel fluidly coupling a bottom end of the first fluid column to a bottom end of the second fluid column. The thermal control unit includes a pump for pumping fluid through the thermal control unit. The thermal control unit includes a heat exchanger adapted to add or remove heat from the fluid, a fluid temperature sensor adapted to sense a temperature of the fluid, and a patient temperature sensor port adapted to receive patient temperature readings from a patient temperature sensor. The thermal control unit includes a controller adapted to control the heat exchanger in order to control the patient's temperature. The thermal control unit includes a return valve adapted to allow ambient air to enter the first fluid column when the return valve is opened and fluid is drained from the thermal control unit. After the return valve is opened, the second internal volume of fluid is adapted to generate a siphon for drawing fluid out of the second hose as the second volume of fluid is pulled downward by gravity.

According to another aspect of the present disclosure, the thermal control unit includes a manifold adapted to control the flow of fluid in the first fluid column and the second fluid column. The return valve is located in the manifold. The thermal control unit includes a supply valve in the manifold that is adapted to allow ambient air to enter the second fluid column when the supply valve is opened. The supply valve is adapted to restrict the incoming airflow and preserve the siphon.

According to still other aspects of the present disclosure, the first fluid port may be a fluid inlet adapted to receive fluid from the first hose when the pump is activated, and the second fluid port may be a fluid outlet adapted to supply fluid to the second hose when the pump is activated.

In some aspects, the drain is positioned at a lower height than the fluid channel, is in fluid communication with the fluid channel, and is adapted to be manually opened and closed by a user.

The return valve, in some aspects, is a pressure activated valve adapted to automatically open after the drain is manually opened and fluid begins draining from the fluid channel.

In some aspects, the return valve is positioned adjacent the top end of the first fluid column.

The thermal control unit, in some aspects, includes a supply valve positioned adjacent the top end of the second fluid column, and the supply valve has a cracking pressure higher than the return valve.

The supply valve, in some aspects, is adapted to allow ambient air to enter the second fluid column when the supply valve is opened and fluid is drained from the thermal control unit.

The supply valve, in some aspects, is adapted to automatically open after the return valve opens.

The thermal control unit, in some aspects, includes a fluid manifold positioned within the fluid channel, and an air separator fluidly coupled to the fluid manifold. The air separator is adapted to vent air from within the fluid manifold to ambient surroundings.

In some aspects, the thermal control unit further includes an air filter in fluid communication with the return valve and adapted to filter the air that enters the first fluid column through the return valve.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being caried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a thermal control system according to one aspect of the present disclosure shown applied to a patient on a patient support apparatus;

FIG. 2 is a perspective view of a thermal control unit of the thermal control system of FIG. 1;

FIG. 3 is a block diagram of the thermal control system of FIG. 1;

FIG. 4 is a perspective view of several internal components of the thermal control unit according to one aspect of the present disclosure;

FIG. 5A is a perspective view of service manifold of the thermal control unit shown from a first perspective;

FIG. 5B is a perspective view of the service manifold of FIG. 5 shown from a different perspective:

FIG. 6 is a perspective view of the service manifold of FIGS. 5A and 5B showing is position relative to other components of the thermal control unit; and

FIG. 7 is a perspective view of an air filter of the thermal control unit.

### DETAILED DESCRIPTION OF THE EMBODIMENTS

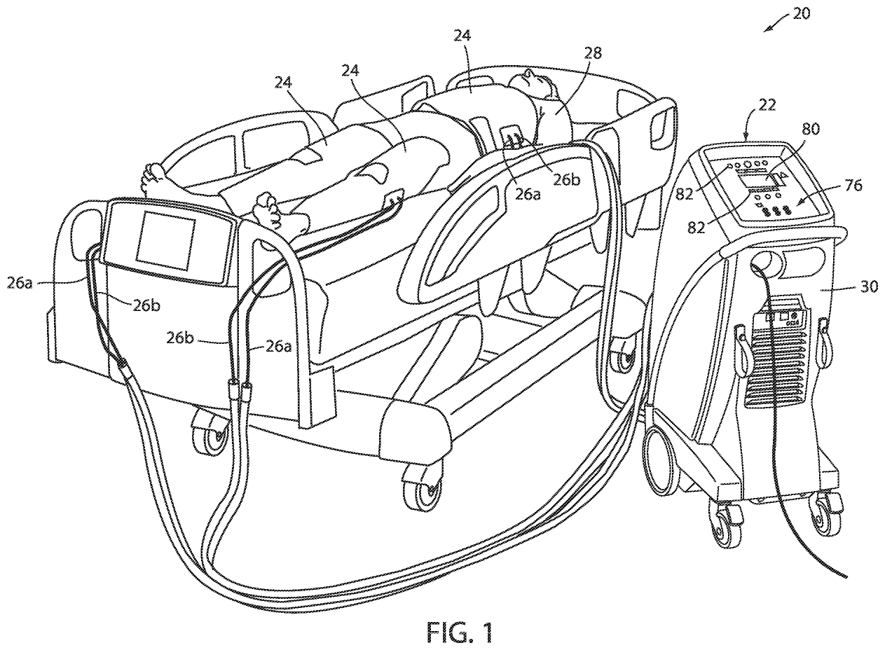

A thermal control system 20 according to one embodiment of the present disclosure is shown in FIG. 1. Thermal control system 20 is adapted to control the temperature of a patient 28, which may involve raising, lowering, and/or maintaining the patient's temperature. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, vests, patches, caps, catheters, or other structures that receive temperature-controlled fluid. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, caps, catheters, etc.) and variations thereof.

Thermal control unit 22 is coupled to thermal pads 24 via a plurality of hoses 26. Thermal control unit 22 delivers temperature-controlled fluid (such as, but not limited to, water or a water mixture) to the thermal pads 24 via the fluid supply hoses 26a. After the temperature-controlled fluid has passed through thermal pads 24, thermal control unit 22 receives the temperature-controlled fluid back from thermal pads 24 via the return hoses 26b.

In the embodiment of thermal control system 20 shown in FIG. 1, three thermal pads 24 are used in the treatment of patient 28. A first thermal pad 24 is wrapped around a patient's torso, while second and third thermal pads 24 are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply hoses 26a, the temperature of the patient 28 can be controlled via the close contact of the pads 24 with the patient 28 and the resultant heat transfer therebetween.

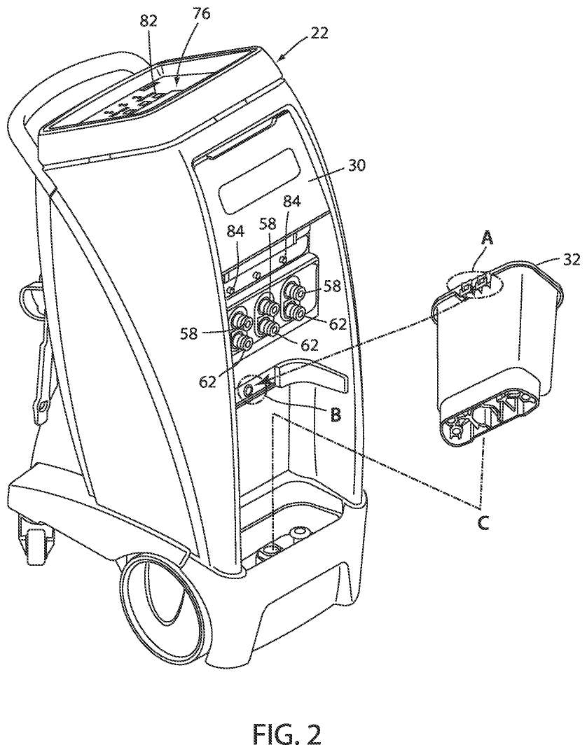

As shown more clearly in FIG. 2, thermal control unit 22 includes a main body 30 to which a removable reservoir 32 may be coupled and uncoupled. Removable reservoir 32 is configured to hold the fluid that is to be circulated through thermal control unit 22 and the one or more thermal pads 24. By being removable from thermal control unit 22, reservoir 32 can be easily carried to a sink or faucet for filling and/or dumping of the water or other fluid. This allows users of thermal control system 20 to more easily fill thermal control unit 22 prior to its use, as well as to drain thermal control unit 22 after use.

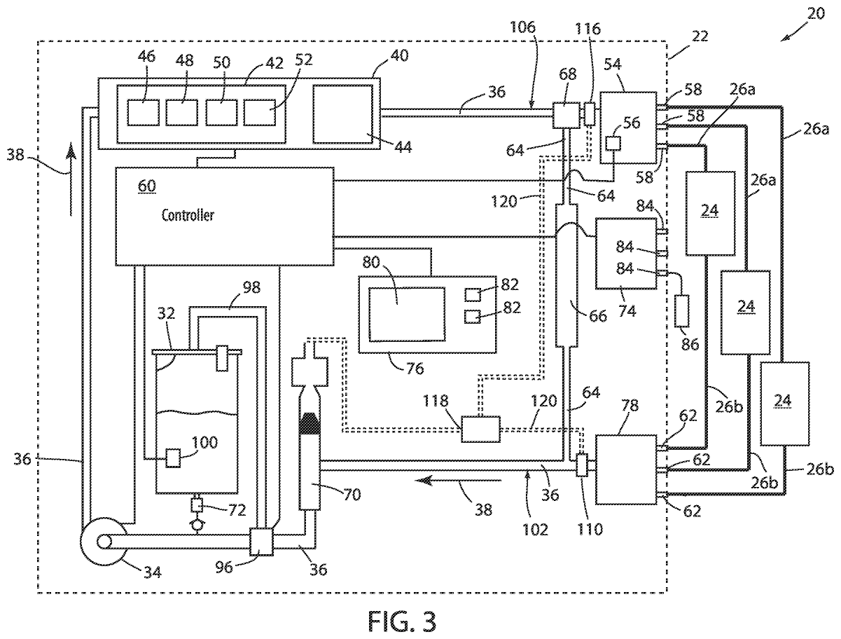

As shown in FIG. 3, thermal control unit 22 includes a pump 34 for circulating fluid through a circulation channel 36. Pump 34, when activated, circulates the fluid through circulation channel 36 in the direction of arrows 38 (clockwise in FIG. 3). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In the embodiment shown in FIG. 3, heat exchanger 40 includes a chiller 42 and a heater 44. Further, in the embodiment shown in FIG. 3, chiller 42 is a conventional vapor-compression refrigeration unit having a compressor 46, a condenser 48, an evaporator 50, an expansion valve (not shown), and a fan 52 for removing heat from the compressor 46. Other types of chillers and/or heaters may be used.

After passing through heat exchanger 40, the circulating fluid is delivered to an outlet manifold 54 having an outlet temperature sensor 56 and a plurality of outlet ports 58. Temperature sensor 56 is adapted to detect a temperature of the fluid inside of outlet manifold 54 and report it to a controller 60. Outlet ports 58 are coupled to supply hoses 26a. Supply hoses 26a are coupled, in turn, to thermal pads 24 and deliver temperature-controlled fluid to the thermal pads 24. The temperature-controlled fluid, after passing through the thermal pads 24, is returned to thermal control unit 22 via return hoses 26b. Return hoses 26b couple to a plurality of inlet ports 62. Inlet ports 62 are fluidly coupled to an inlet manifold 78 inside of thermal control unit 22.

Thermal control unit 22 also includes a bypass line 64 fluidly coupled to outlet manifold 54 and inlet manifold 78 (FIG. 3). Bypass line 64 allows fluid to circulate through circulation channel 36 even in the absence of any thermal pads 24 or hoses 26a being coupled to any of outlet ports 58. In the illustrated embodiment, bypass line 64 includes an optional filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both. In some embodiments, filter 66 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 64 is controllable by way of a bypass valve 68 positioned at the intersection of bypass line 64 and outlet manifold 54 (FIG. 3). When open, bypass valve 68 allows fluid to flow through circulation channel 36 to outlet manifold 54, and from outlet manifold 54 to the connected thermal pads 24. When closed, bypass valve 68 stops fluid from flowing to outlet manifold 54 (and thermal pads 24) and instead diverts the fluid flow along bypass line 64. In some embodiments, bypass valve 68 may be controllable such that selective portions of the fluid are directed to outlet manifold 54 and along bypass line 64. In some embodiments, bypass valve 68 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference.

The incoming fluid flowing into inlet manifold 78 from inlet ports 62 and/or bypass line 64 travels back toward pump 34 and into an air remover 70. Air remover 70 includes any structure in which the flow of fluid slows down sufficiently to allow air bubbles contained within the circulating fluid to float upwardly and escape to the ambient surroundings. In some embodiments, air remover 70 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 70, the circulating fluid flows past a valve 72 positioned beneath fluid reservoir 32. Fluid reservoir 32 supplies fluid to thermal control unit 22 and circulation channel 36 via valve 72, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 72, the circulating fluid travels to pump 34 and the circuit is repeated.

Controller 60 of thermal control unit 22 is contained within main body 30 of thermal control unit 22 and is in electrical communication with pump 34, heat exchanger 40, outlet temperature sensor 56, bypass valve 68, a patient temperature module 74, and a user interface 76. Controller 60 includes any and all electrical circuitry and components necessary to carry out the functions and algorithms described herein, as would be known to one of ordinary skill in the art. Generally speaking, controller 60 may include one or more microcontrollers, microprocessors, and/or other programmable electronics that are programmed to carry out the functions described herein. It will be understood that controller 60 may also include other electronic components that are programmed to carry out the functions described herein, or that support the microcontrollers, microprocessors, and/or other electronics. The other electronic components include, but are not limited to, one or more field programmable gate arrays, systems on a chip, volatile or non-volatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

User interface 76, which may be implemented as a control panel or in other manners, allows a user to operate thermal control unit 22. User interface 76 communicates with controller 60 and includes a display 80 and a plurality of dedicated controls 82. Display 80 may be implemented as a touch screen, or, in some embodiments, as a non-touch-sensitive display. Dedicated controls 82 may be implemented as buttons, switches, dials, or other dedicated structures. In any of the embodiments, one or more of the functions carried out by a dedicated control 82 may be replaced or supplemented with a touch screen control that is activated when touched by a user. Alternatively, in any of the embodiments, one or more of the controls that are carried out via a touch screen can be replaced or supplemented with a dedicated control 82 that carries out the same function when activated by a user.

Through either dedicated controls 82 and/or a touch screen display (e.g. display 80), user interface 76 enables a user to turn thermal control unit 22 on and off, select a mode of operation, select a target temperature for the fluid delivered to thermal pads 24, select a patient target temperature, and control other aspects of thermal control unit 22. In some embodiments, user interface 76 may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 82.

In those embodiments where user interface 76 allows a user to select from different modes for controlling the patient's temperature, the different modes include, but are not limited to, a manual mode and an automatic mode, both of which may be used for cooling and heating the patient. In the manual mode, a user selects a target temperature for the fluid that circulates within thermal control unit 22 and that is delivered to thermal pads 24. Thermal control unit 22 then makes adjustments to heat exchanger 40 in order to ensure that the temperature of the fluid exiting supply hoses 26a is at the user-selected temperature.

Another one of the modes is an automatic mode. When the user selects the automatic mode, the user selects a target patient temperature, rather than a target fluid temperature. After selecting the target patient temperature, controller 60 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, thermal control unit 22 utilizes patient temperature module 74. Patient temperature module 74 includes one or more patient temperature sensor ports 84 (FIGS. 2 & 3) that are adapted to receive one or more conventional patient temperature sensors or probes 86. The patient temperature sensors 86 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant. In other embodiments, different types of sensors may be used with thermal control unit 22. Regardless of the specific type of patient temperature sensor used in thermal control system 20, each temperature sensor 86 is connected to a patient temperature sensor port 84 positioned on thermal control unit 22. Patient temperature sensor ports 84 are in electrical communication with controller 60 and provide current temperature readings of the patient's temperature.

Controller 60, in some embodiments, controls the temperature of the circulating fluid using closed-loop feedback from temperature sensor 56. That is, controller 60 determines (or receives) a target temperature of the fluid, compares it to the measured temperature from sensor 56, and issues a command to heat exchanger 40 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 60 multiplies the fluid temperature error by a proportional constant, determines the derivative of the fluid temperature error over time and multiplies it by a derivative constant, and determines the integral of the fluid temperature error over time and multiplies it by an integral constant. The results of each product are summed together and converted to a heating/cooling command that is fed to heat exchanger 40 and tells heat exchanger 40 whether to heat and/or cool the circulating fluid and how much heating/cooling power to use.

When thermal control unit 22 is operating in the automatic mode, controller 60 may use a second closed-loop control loop that determines the difference between a patient target temperature 88 and a measured patient temperature 90. The patient target temperature 88 is input by a user of thermal control unit 22 using user interface 76. Measured patient temperature 90 comes from a patient temperature sensor 86 coupled to one of patient temperature sensor ports 84 (FIG. 3). Controller 60 determines the difference between the patient target temperature 88 and the measured patient temperature 90 and, in some embodiments, uses the resulting patient temperature error value as an input into a conventional PID control loop. As part of the PID loop, controller 60 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 60 may utilize one or more PI loops, PD loops, and/or other types of control equations. In some embodiments, the coefficients used with the control loops may be varied by controller 60 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 60 implements the loop(s) multiple times a second in at least one embodiment, although it will be understood that this rate may be varied widely. After controller 60 has output a heat/cool command to heat exchanger 40, controller 60 takes another patient temperature reading (from sensor 86) and/or another fluid temperature reading (from sensor 56) and re-performs the loop(s). The specific loop(s) used, as noted previously, depends upon whether thermal control unit 22 is operating in the manual mode or automatic mode.

It will also be understood by those skilled in the art that the output of any control loop used by thermal control unit 22 may be limited such that the temperature of the fluid delivered to thermal pads 24 never strays outside of a predefined maximum and a predefined minimum. The minimum temperature is designed as a safety temperature and may be set to about four degrees Celsius, although other temperatures may be selected. The predefined maximum temperature is also implemented as a safety measure and may be set to about forty degrees Celsius, although other values may be selected.

In the embodiment shown in FIG. 3, thermal control unit 22 also includes a reservoir valve 96 that is adapted to selectively move fluid reservoir 32 into and out of line with circulation channel 36. Reservoir valve 96 is positioned in circulation channel 36 between air remover 70 and valve 72, although it will be understood that reservoir valve 96 may be moved to different locations within circulation channel 36. Reservoir valve 96 is coupled to circulation channel 36 as well as a reservoir channel 98. When reservoir valve 96 is open, fluid from air remover 70 flows along circulation channel 36 to pump 34 without passing through reservoir 32 and without any fluid flowing along reservoir channel 98. When reservoir valve 96 is closed, fluid coming from air remover 70 flows along reservoir channel 98, which feeds the fluid into reservoir 32. Fluid inside of reservoir 32 then flows back into circulation channel 36 via valve 72. Once back in circulation channel 36, the fluid flows to pump 34 and is pumped to the rest of circulation channel 36 and thermal pads 24 and/or bypass line 64. In some embodiments, reservoir valve 96 is either fully open or fully closed, while in other embodiments, reservoir valve 96 may be partially open or partially closed. In either case, reservoir valve 96 is under the control of controller 60.

Thermal control unit 22 also includes a reservoir temperature sensor 100. Reservoir temperature sensor 100 reports its temperature readings to controller 60. When reservoir valve 96 is open, the fluid inside of reservoir 32 stays inside of reservoir 32 (after the initial drainage of the amount of fluid needed to fill circulation channel 36 and thermal pads 24). This residual fluid is substantially not affected by the temperature changes made to the fluid within circulation channel 36 as long as reservoir valve 96 remains open. This is because the residual fluid that remains inside of reservoir 32 after circulation channel 36 and thermal pads 24 have been filled does not pass through heat exchanger 40 and remains substantially thermally isolated from the circulating fluid. Two results flow from this: first, heat exchanger 40 does not need to expend energy on changing the temperature of the residual fluid in reservoir 32, and second, the temperature of the circulating fluid in circulation channel 36 will deviate from the temperature of the residual fluid as the circulating fluid circulates through heat exchanger 40.

Thermal control unit 22 further includes a supply valve 116, a return valve 110, a first fluid column 102, a second fluid column 106, and an air filter 118 (FIG. 3). Although first fluid column 102 and second fluid column 106 are depicted in a horizontal orientation in FIG. 3, it will be understood that FIG. 3 is merely a block diagram, and that in the actual thermal control unit, first and second fluid columns 102 and 106 are oriented generally vertically so that gravity exerts a downward force on the fluid, urging the fluid within the columns toward the floor. As will be discussed in greater detail below, supply valve 116 and return valve 110 are coupled to a source of ambient air via air lines 120. Air lines 120 are fluidly coupled to air filter 118, and air filter 118 includes an opening (not shown) that allows it to draw in ambient air, filter it, and supply it to valves 110 and 116.

Controller 60 utilizes a temperature control algorithm to control reservoir valve 96 that, in some embodiments, is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 60 utilizes a different control algorithm. In still other embodiments, thermal control unit 22 is modified to omit reservoir valve 96, reservoir channel 98, and reservoir temperature sensor 100. Thermal control unit 22 may also be modified such that reservoir 32 is always in the path of circulation channel 36. Still other modifications are possible.

Although other designs may be used, some suitable examples thermal pads incorporating temperature sensors that may be used for detecting peripheral temperature are found in commonly assigned U.S. patent application Ser. No. 62/425,813 filed Nov. 23, 2016, by inventors Gregory Taylor et al. and entitled THERMAL SYSTEM, as well as commonly assigned U.S. patent application Ser. No. 15/675,066 filed Aug. 11, 2017, by inventor James K. Galer and entitled THERMAL SYSTEM, the complete disclosures of both of which are hereby incorporated by reference in their entirety herein. Regardless of whether the peripheral temperature sensor(s) are incorporated into a thermal pad 24 or not, the outputs from the temperature sensor(s) are fed to controller 60. In some embodiments, the sensor outputs are fed to controller 60 via cables coupled from the temperature sensors to patient temperature input ports 84. It will be understood that thermal control unit 22 can include more patient temperature input ports 84 than the three shown in FIG. 2.

It will also be understood that any of the thermal control units disclosed herein may be modified to additionally operate in conjunction with one or more auxiliary sensors used to sense one or more non-temperature patient parameters. When so modified, any of the thermal control units disclosed herein may utilize the auxiliary sensors in any of the manners, and using any of the structures and/or algorithms, disclosed in commonly assigned U.S. patent application Ser. No. 62/610,327 filed Dec. 26, 2017, by inventors Gregory S. Taylor et al. and entitled THERMAL SYSTEM WITH PATIENT SENSOR(S), the complete disclosure of which is incorporated herein by reference.

Any of the thermal control units disclosed herein may also or alternatively be modified to incorporate any of the temperature overshoot reduction methods, structures, and/or algorithms disclosed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference. Additionally or alternatively, any of the thermal control units disclosed herein may use any of the data and algorithms disclosed in U.S. patent application Ser. No. 62/610,334 filed Dec. 26, 2017, by inventors Christopher Hopper et al. and entitled THERMAL CONTROL SYSTEM when determining when a patient's core temperature will reach its temperature, and/or when to transition from heating the circulating fluid to cooling the circulating fluid, and vice versa, in order to reduce overshoot. The '334 application is hereby incorporated herein by reference in its entirety.

It will be understood by those skilled in the art that the thermal control unit 22 can be modified in many different ways. Some possible alternatives are outlined in commonly assigned U.S. patent application Ser. No. 16/222,004 filed Dec. 17, 2018, by inventors Gregory Taylor et al. and entitled THERMAL SYSTEM WITH GRAPHICAL USER INTERFACE, the complete disclosure of which is incorporated herein by reference.

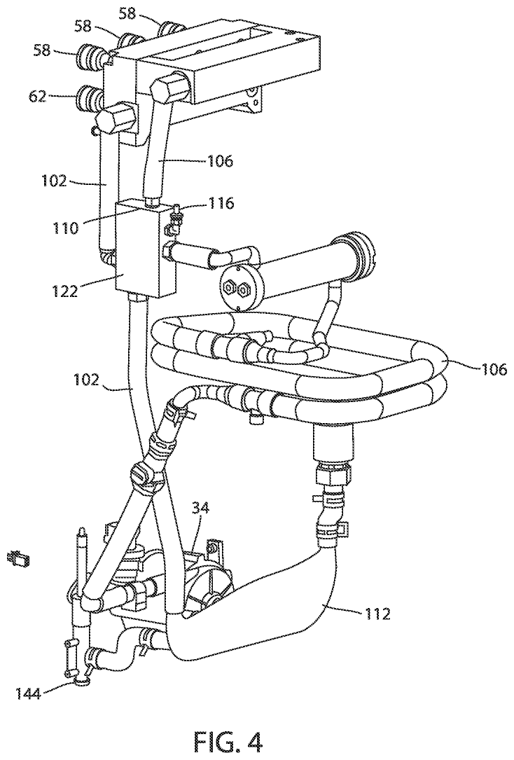

Several of the internal components of the thermal control unit 22 according to one aspect are shown in FIG. 4. These components include first fluid column 102, which is fluidly coupled to inlet ports 62 at a top end of the first fluid column 102. The first fluid column 102 includes a first internal volume of fluid. The first fluid column 102 is the return column through which fluid initially flows after it is returned from the thermal pad(s) 24. The first fluid column 102 may alternately be referred to as the return circuit. The inlet ports 62 at the top of first fluid column 102 allow multiple hoses to be connected to the first fluid column 102 as shown in FIG. 4.

The thermal control unit 22 includes a second fluid column 106 fluidly coupled at its top end to outlet ports 58. The second fluid column 106 includes a second internal volume of fluid. The second internal volume is greater than the first internal volume. The second fluid column 106 is a supply column connected to the outlet ports 58. The second fluid column 106 may alternately be referred to as the supply circuit. The outlet ports 58 at the top end of second fluid column 106 allow multiple hoses to be connected to the second fluid column 106 as shown in FIG. 4.

A fluid manifold 112 fluidly couples a bottom end of the first fluid column 102 to a bottom end of the second fluid column 106. Fluid manifold 112 also includes a connection (not shown) to the bottom end of removable reservoir 32 so that fluid positioned inside of the reservoir 32 is initially drawn by gravity out of the removable reservoir and into the fluid manifold 112. When pump 34 is activated, the fluid inside of manifold 112 is pumped upwards through second fluid column 106 and additional fluid from reservoir 32 flows into manifold 112 to replace the pumped fluid. This filling of manifold 112 with fluid from reservoir 32 continues until the air inside of circulation channel 36 is removed and replaced with the pumped liquid.

Thermal control unit 22 includes a drain 114 positioned at a lower height than the fluid manifold 112 and in fluid communication with the fluid manifold 112. The drain 114 may be adapted to be manually opened and closed by a user. When the drain 114 is open, fluid can flow out of the fluid manifold 112, and thereby out of the thermal control unit 22. In one aspect, the drain 114 can be adapted to be remotely opened and closed. In one aspect, the drain 114 may be automatically opened and closed when the thermal control unit 22 is to be drained. The first hose and the second hose can be placed substantially horizontally during the draining process. Air separator 70 may be fluidly coupled to the fluid manifold and adapted to vent air from within the fluid manifold to ambient surroundings.

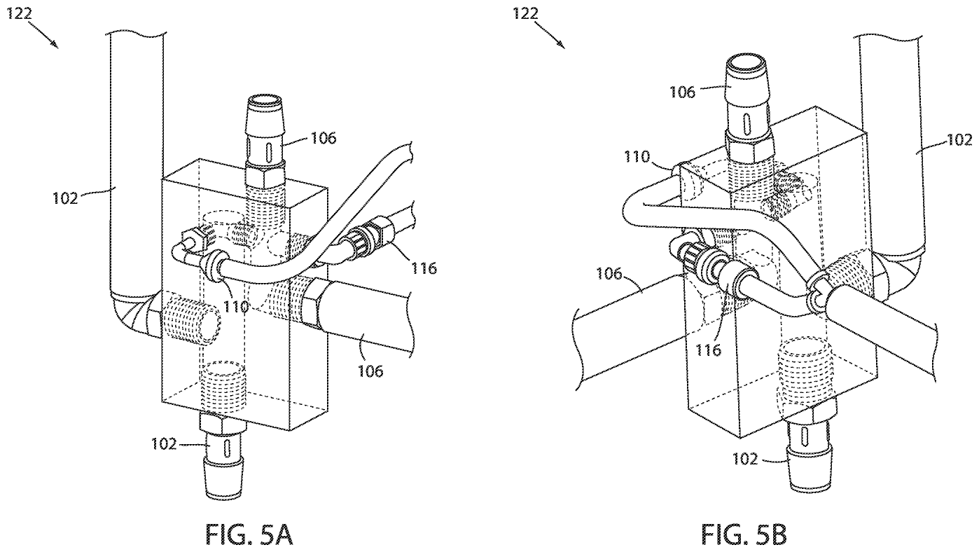

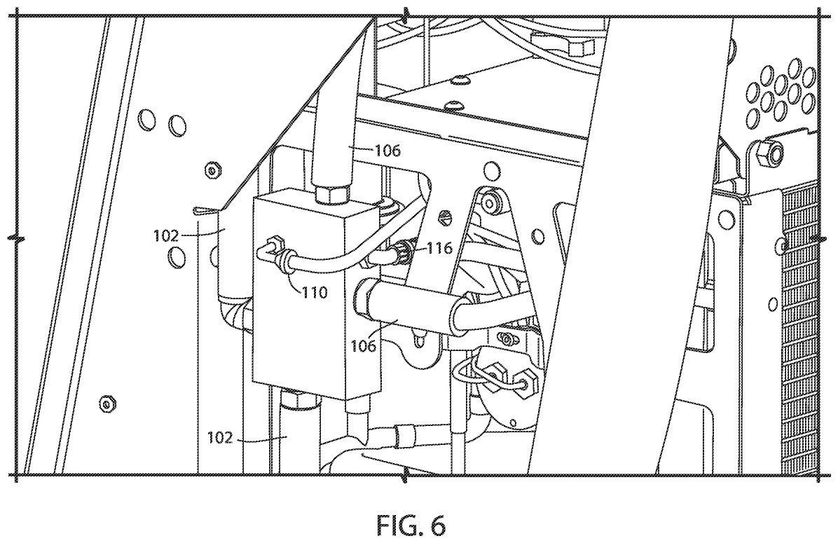

Thermal control unit 22 may include a service manifold 122 (FIG. 4) that includes return valve 110 and supply valve 116. The service manifold 122 can be adapted to control the flow of fluid in the first fluid column 102 and the second fluid column 106 when thermal control unit 22 is drained. A close-up of the service manifold 122 according to one aspect is shown in FIGS. 5A-5B and 6. In one aspect, the thermal control unit 22 may include the return valve 110 and the supply valve 116 without the service manifold 122. In another aspect, the thermal control unit 22 can include the service manifold 122 and either the return valve 110 or the supply valve 116. In another aspect, the thermal control unit 22 may include the return valve 110 without the supply valve 116 or the service manifold 122. In yet another aspect, the thermal control unit 22 can include the supply valve 116 without the return valve 110 or the service manifold 122. In one aspect, the return valve 110 may be located in the service manifold 122. In another aspect, the return valve 110 can be coupled to the service manifold 122.

As depicted in FIG. 4, the return valve 110 may be positioned adjacent the top end of the first fluid column 102. The return valve 110 may be adapted to allow ambient air to enter the first fluid column 102 when the return valve 110 is opened and fluid is drained from the thermal control unit 22. In one aspect, the return valve 110 may allow for no air flow restriction into the system. After the return valve 110 is opened, the second internal volume of fluid in the second fluid column 106 is adapted to generate a siphon for drawing fluid out of the hoses coupled to the thermal control unit 22 as the second volume of fluid is pulled downward by gravity. Put another way, the mass imbalance between the first internal volume of first column 102 and the second internal volume of second column 106 generates a siphon that pulls fluid inside of the hoses toward supply column 106 and down toward the fluid manifold 112, from which it eventually exits through the drain 114. The siphon continues until one of the hoses drains completely. The siphon is broken when one of the hoses is empty because both ends of all remaining hoses are vented to the atmosphere through the empty hose. Siphoning the fluid through the thermal control unit 22 allows the residual fluid in the hoses to be minimized.

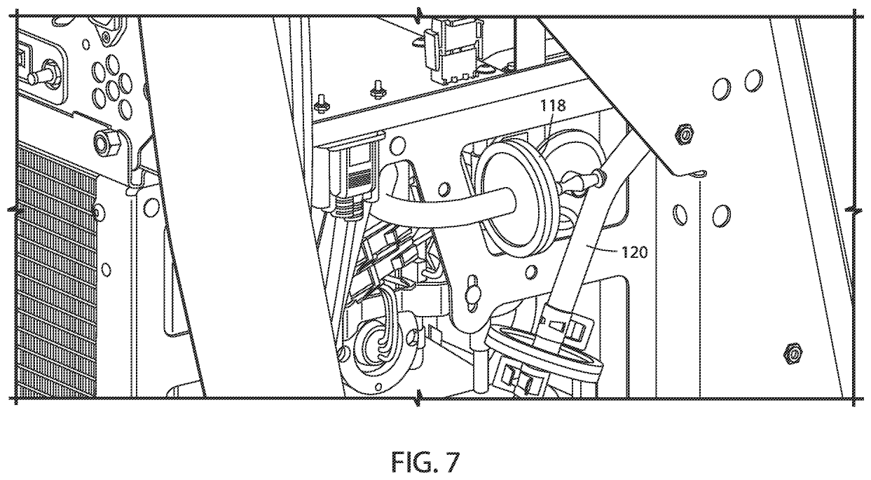

In one aspect, the return valve 110 is a pressure activated valve adapted to automatically open after the drain 114 is opened and fluid begins draining from the fluid manifold 112. The fluid in the first fluid column 102 can completely drain to the fluid manifold 112 because the return valve 110 provides robust venting to the atmosphere. In one aspect, air filter 118 is in fluid communication with the return valve 110. The airfilter 118 may be adapted to filter the air that enters the first fluid column 102 through the return valve 110. An exemplary configuration of the air filter 118 is shown in FIG. 7. In one aspect, the air filter 118 may be a high efficiency particulate air (“HEPA”) filter. In an alternative aspect, return valve 110 can be an electronically-controlled valve that is opened by the controller 60 when the drain 114 is opened. However, in the embodiment primarily described herein, the weight of the fluid inside first column 102 is sufficient to automatically crack open return valve 110 when drain 114 is opened, thereby automatically allowing filtered air to enter into first fluid column 102 at the location of return valve 110.

Returning to FIG. 4, the supply valve 116 may be adapted to allow ambient air to enter the second fluid column 106 when the supply valve 116 is opened and fluid is drained from the thermal control unit 22. The supply valve 116 may be adapted to restrict the incoming airflow into the thermal control unit 22 and preserve the siphon through the entire draining process. In one aspect, the supply valve 116 may be positioned adjacent a top end of the second fluid column 106. As the fluid falls from the second fluid column 106 to the fluid manifold 112, fluid within the hoses flows is sucked toward the second fluid column 106. The fluid may then enter the fluid manifold 112 from the second fluid column 106 and leave the thermal control unit 22 through the drain 114. In one aspect, the supply valve 116 may be located in the service manifold 122. In an alternate aspect, the supply valve 116 can be coupled to the service manifold 122. In one aspect, the supply valve 116 can have a higher cracking pressure than the return valve 110. The supply valve 116 may be adapted to automatically open after the return valve 110 opens. In one aspect, there may be a predetermined delay period between when the return valve 110 opens and when the supply valve 116 opens. In one aspect, the supply valve 116 is a pressure activated valve that is adapted to automatically open when drain 114 is opened. In another aspect, the supply valve 116 can be opened by the controller 60.

As shown in FIG. 7, the air filter 118 filters the air that enters the first fluid column 102 and the second fluid column 106 through valves 110 and 116. In one aspect, the air filter 118 may only filter the air that enters the second fluid column 106. In one aspect, the supply valve 116 may have a smaller aperture than the return valve 110. A smaller aperture allows the supply valve 116 to have a slower airflow and allow less air to enter the thermal control unit 22 than the return valve 110. In one aspect, the supply valve 116 can include an orifice sized to restrict incoming airflow and preserve the siphon. The orifice may maintain the pressure differential between the first fluid column 102 and the second fluid column 106. The orifice may slow the draining of the fluid to preserve the mass imbalance. In one aspect, the orifice may be designed to generate ten inches of water pressure while maintaining an adequate flow. The supply valve 116 allows for the thermal control unit 22 to be completely drained after the hoses are empty. In one aspect, the supply valve 116 allows the first fluid column 102 and the second fluid column 106 to empty even after the first hose and the second hose are empty and the siphon is broken.

Various other alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A thermal control unit for controlling a patient's temperature during a thermal therapy session, the thermal control unit comprising:
a first fluid port adapted to fluidly couple to a first hose;
a second fluid port adapted to fluidly couple to a second hose;
a first fluid column having a top end fluidly coupled to the first fluid port, the first fluid column including a first internal volume;
a second fluid column having a top end fluidly coupled to the second fluid port, the second fluid column having a second internal volume greater than the first internal volume;
a fluid channel fluidly coupling a bottom end of the first fluid column to a bottom end of the second fluid column;
a pump for pumping fluid through the thermal control unit;
a heat exchanger adapted to add or remove heat from the fluid;
a fluid temperature sensor adapted to sense a temperature of the fluid;
a patient temperature sensor port adapted to receive patient temperature readings from a patient temperature sensor;
a controller adapted to control the heat exchanger in order to control the patient's temperature; and
a return valve adapted to allow ambient air to enter the first fluid column when the return valve is opened and fluid is drained from the thermal control unit, wherein after the return valve is opened, the second internal volume of fluid is adapted to generate a siphon for drawing fluid out of the second hose as the second volume of fluid is pulled downward by gravity.

2. The thermal control unit of claim 1 wherein the first fluid port is a fluid inlet adapted to receive fluid from the first hose when the pump is activated, and the second fluid port is a fluid outlet adapted to supply fluid to the second hose when the pump is activated.

3. The thermal control unit further including a drain positioned at a lower height than the fluid channel and in fluid communication with the fluid channel, the drain adapted to be manually opened and closed by a user.

4. The thermal control unit of claim 3 wherein said return valve is a pressure activated valve adapted to automatically open after the drain is manually opened and fluid begins draining from the fluid channel.

5. The thermal control unit of claim 4 wherein the return valve is positioned adjacent the top end of the first fluid column.

6. The thermal control unit of claim 5 further comprising a supply valve positioned adjacent the top end of the second fluid column, said supply valve having a cracking pressure higher than the return valve.

7. The thermal control unit of claim 6 wherein the supply valve is adapted to allow ambient air to enter the second fluid column when the supply valve is opened and fluid is drained from the thermal control unit.

8. The thermal control unit of claim 7 wherein the supply valve is adapted to automatically open after the return valve opens.

9. The thermal control unit of claim 1 further comprising a fluid manifold positioned within the fluid channel, and an air separator fluidly coupled to the fluid manifold, the air separator adapted to vent air from within the fluid manifold to ambient surroundings.

10. The thermal control unit of claim 1 further comprising an air filter in fluid communication with the return valve and adapted to filter the air that enters the first fluid column through the return valve.

11. A thermal control unit for controlling a patient's temperature during a thermal therapy session, the thermal control unit comprising:
a first fluid port adapted to fluidly couple to a first hose;
a second fluid port adapted to fluidly couple to a second hose;
a first fluid column having a top end fluidly coupled to the first fluid port, the first fluid column including a first internal volume;
a second fluid column having a top end fluidly coupled to the second fluid port, the second fluid column having a second internal volume greater than the first internal volume;
a fluid channel fluidly coupling a bottom end of the first fluid column to a bottom end of the second fluid column;
a pump for pumping fluid through the thermal control unit;
a heat exchanger adapted to add or remove heat from the fluid;
a fluid temperature sensor adapted to sense a temperature of the fluid;
a patient temperature sensor port adapted to receive patient temperature readings from a patient temperature sensor;
a controller adapted to control the heat exchanger in order to control the patient's temperature;
a return valve adapted to allow ambient air to enter the first fluid column when the return valve is opened and fluid is drained from the thermal control unit, wherein after the return valve is opened, the second internal volume of fluid is adapted to generate a siphon for drawing fluid out of the second hose as the second volume of fluid is pulled downward by gravity; and
a supply valve adapted to allow ambient air to enter the second fluid column when the supply valve is opened, wherein the supply valve is adapted to restrict the incoming airflow and preserve the siphon.

12. The thermal control unit of claim 11 wherein the supply valve is adapted to open after the return valve.

13. The thermal control unit of claim 11 wherein the supply valve has a higher cracking pressure than the return valve.

14. The thermal control unit of claim 11 wherein the supply valve has a larger orifice than the return valve.

15. The thermal control unit of claim 11 further including a drain positioned at a lower height than the fluid channel and in fluid communication with the fluid channel, the drain adapted to be manually opened and closed by a user.

16. The thermal control unit of claim 15 wherein said return valve is a pressure activated valve adapted to automatically open after the drain is manually opened and fluid begins draining from the fluid channel.

17. The thermal control unit of claim 11 further comprising a manifold positioned within the fluid channel, and an air separator fluidly coupled to the manifold, the air separator adapted to vent air from within the manifold to ambient surroundings.

18. The thermal control unit of claim 11 wherein the supply valve allows the first fluid column and the second fluid column to be drained when the first hose and the second hose are empty.

19. The thermal control unit of claim 11 wherein the return valve is positioned adjacent a top end of the first fluid column.

20. The thermal control unit of claim 11 wherein the supply valve is positioned adjacent a top end of the second fluid column.
